(19) United States
(12) Patent Application Publication
(10) Pub. No.: US 2026/0151028 A1
(43) Pub. Date: Jun. 4, 2026

(54) DEVICE AND METHODS FOR IMPROVING VISUAL BLURRINESS

(71) Applicant: BAR-ILAN UNIVERSITY, Ramat Gan (IL)

(72) Inventors: Uri POLAT, Ramat Gan (IL); Maria LEV, Herzliya (IL)

(73) Assignee: BAR-ILAN UNIVERSITY, Ramat Gan (IL)

(21) Appl. No.: 18/706,395

(22) PCT Filed: Oct. 30, 2022
(86) PCT No.: PCT/IL2022/051147

§ 371 Date: May 1, 2024

Related U.S. Application Data
(60) Provisional application No. 63/274,065, filed on Nov. 1, 2021.

(51) Int. Cl. A61B 3/00; A61B 3/032

(52) U.S. Cl. CPC A61B 3/0058; A61B 3/032

(57) ABSTRACT

A device and methods thereof to directly interact with Blurred Image Processing (BIP) and to enable optimal and comfortable functional vision, thus enhance visual abilities. The observer is presented, via the device, with a set of stimuli, identical except for their respective blurs, and the observer's task is to identify the stimulus with the larger blur, or alternatively the smaller.

### BACKGROUND OF THE INVENTION

The human eye may be described as optical device aiming to project the image on the retina. The retina acts as an encoding system that transform the image to digitized local information (pixels) by means of neuronal code that is transferred to the brain. The main process of vision is performed in the brain. About 50% of the cortical area is involved in vision processing. The purpose of the visual processing is to decode the retinal information and perform interpretation of the best possible solution. This process involves perception and recognition of the visual information, conditions that capture the real functional vision and not the quality of the optical correction. Therefore, even best optical correction may pose processing load and inconvenience in the task of perceiving and recognition of the retinal image.

Image input is defined by retinal projection of an image, by means of neuronal code that is transferred to the brain. Image perception is defined as visual processing in the brain of such transferred retinal information, involving the best possible perception, recognition and interpretation. Retinal defocus (out of focus) causes blurred image input to the visual cortex. Such Blurred Image Input elicits weaker and slower neuronal responses, with a consequent Blurred Image Perception, resulting in a reduction in visual acuity (VA) and contrast sensitivity.

Others describe visual acuity tests that were typically conducted using letters, lines or other objects. Such diversity contributed to the large variability (demonstrated in FIGS. 1A-1D). The classic measuring of visual acuity by finding the minimal letter/gap that the subject can see, relied on the assumption that the subject's visual acuity limits by the blur's size.

Blur is difficult to define. In the narrower technical context of vision, optics, and imaging, blurring generally means a smearing of an image, through some amount of low-pass filtering. Blur is an important attribute of human vision and has large interest in the role of blur as a visual cue. Over the last decades, it has become common to analyze the early parts of the visual system as an imaging system, incorporating various optical and neural filtering operations. This in turn has led to questions about the nature of blur in vision and its effect on visual tasks such as visual acuity.

### SUMMARY OF THE INVENTION

According to some embodiments of the invention, a new device and methods thereof directly interact with Blurred Image Processing (BIP) to enable optimal and comfortable functional vision, thus enhance visual abilities.

According to some embodiments, the observer is presented, via the new device, with a set of stimuli, identical except for their respective blurs, and the observer's task is to identify the stimulus with the larger blur (or alternatively the smaller).

According to some embodiments, the device is configured to present an observer a set of stimuli images, identical images except for their respective blur level. One of the images is the “reference” image, i.e., a clear image, and the other/s comprise/s a “test” blur, which may be considered as the reference image plus an added blur level/s. The observer's task is to identify the stimuli with the blur. The procedure is repeated over a series of trials, while the blur level is fixed (e.g., decreased) and the amount of added blur is varied until determining the current threshold of the added blur, that is, the amount of added blur at which the observer is correct some specified percentage of the time, for a specific stage of the training. Over the course of training, the difficulty level of blur is increased (meaning that the blur level is decreased therefore harder to identify) until the observer reached enough correct responses, again at the current training stage. This design is repeated until the observer's improvement, over several training stages has reached to a saturation.

According to some embodiments, the set of stimuli images selected overcome the variability of the perception of sharpness have no sharpness cues, such as edges.

According to Some Embodiments of the Invention a New Method is Provided for Presenting Images to a User, the Method Comprising Processor Implemented Method Steps of:
A. displaying, at a given time-step N (N=K, 1≤K≤Kₛ) of a session S of time steps, at least one reference image and at least one blurred image to the user; wherein both the reference and the blurred image comprise the same original image with different predetermined levels of blurriness applied thereon Br, Bb, selected per said session S; and wherein Bᵣ<B_{b};
B. receiving and/collecting, at said given time-step (N=K), the user's response to the currently displayed images, wherein the response comprises the user's indication of the reference image and/or the blurred image;
C. analyzing correctness of the user's response, received at said given time-step (N=K) and any former time-step/s (N<K) of said session S;
D. repeating method steps A to C with following time-steps (K=K+1) of said session S, a predetermined number of times Kₛ for said session S and/or until the correctness analysis of method step C has reached a predetermined correctness threshold.

According to some embodiments, the method further comprising method step E of repeating method step D, with at least one following session (S=S+1) of time-steps (N=K, 1≤K≤Kₛ), while applying the reference image and/or the blurred image with a different level/s of blurriness (Bᵣ<B_{b}) for said following session.

According to some embodiments, the new level/s of predetermined blurriness (Bᵣ and/or B_{b}) is/are selected respective to the analysis results of the former method step/s C.

According to some embodiments, the method further comprises repeating method step E a predetermined number of times and/or until a comparison analysis of all former sessions has reached a predetermined analysis threshold.

According to some embodiments, method step E is provided with a predetermined time gap.

According to some embodiments, the new level/s of predetermined blurriness (Bᵣ and/or B_{b}) is/are selected with a smaller gap between Bᵣ and B_{b}.

According to some embodiments, method step E comprises selecting B_{b} which is the user's current blurriness threshold (B_{b}=Bₜₕ).

According to some embodiments, the method further comprising testing the user to determine one's current blurriness threshold Bₜₕ, at which the user's responses are correct a predetermined percentage of time steps.

According to some embodiments, the user is provided with a real-time feedback of one's correctness, at said given time step (N=K).

According to some embodiments, the displaying of the reference vs. blurred images is provided either simultaneously or sequentially.

According to some embodiments, the original image comprises a round shape.

According to some embodiments, the original image comprises no sharp edges.

According to some embodiments, the blurred level B is set by a Gaussian variable Sigma.

According to some embodiments, both the reference and blurred images, at each time step (N=K), are displayed with same predetermined features selected from: background, contrast, colors, brightness, other images.

According to some embodiments, the original image changes between the different time steps and/or wherein the predetermined selected features change between the different time steps.

According to some embodiments, a new method is provided for presenting images to a user, the method comprising processor implemented method steps of:
a. displaying, at a given time-step N (N=K, 1<K≤Kₛ) of a session of time steps, at least one test image; wherein each of the test images comprises its own original image with a predetermined level of blurriness applied thereon Bₜ, selected per said time step (N=K);
b. receiving and/or collecting, at said given time-step (N=K), the user's response to the currently displayed images, wherein the response comprises the user's indication whether the at least one test image is clear or blurred;
c. analyzing the user's response, received at said given time-step (N=K) and any former time-step/s (N<K) of said session;
d. repeating method steps a to c with following time-steps (K=K+1) of said session, a predetermined number of times Kₛ for said session and/or until the analysis of method step c has reached a predetermined threshold; wherein the selected Bₜ for the following time-step is based on the analysis of step c.

According to some embodiments, method step d is provided with a predetermined time gap.

According to some embodiments, method step d comprises selecting Bₜ which is the user's current blurriness threshold (Bₜ=Bₜₕ).

According to some embodiments, the method further comprising testing the user to determine one's current blurriness threshold Bₜₕ, at which the user's responses are correct a predetermined percentage of time steps.

According to some embodiments, the user is provided with a real-time feedback of one's correctness, at said given time step (N=K).

According to some embodiments, the original/s image comprise a round shape.

According to some embodiments, the original/s image comprise no sharp edges.

According to some embodiments, the blurred level B is set by a Gaussian variable Sigma.

According to some embodiments, in the case of more than one test image, all test images at said time step (N=K), are displayed with same predetermined features selected from: background, contrast, colors, brightness, other images.

According to some embodiments, the original image changes between the different time steps and/or wherein the predetermined selected features change between the different time steps.

According to some embodiments of the invention a new method is provided for treating a subject afflicted with Myopia and/or Age-related Macular Degeneration (AMD), the method comprising presenting images to the subject according to any one of the above-mentioned method steps.

According to some embodiments, a new device is provided configured to present images to a user, comprising:
at least one processor configured to implement the method steps according to according to any one of the above mentioned embodiments;
at least one display-device, configured to display the images to the user; and
at least one input-device, configured to collect and interpret, at the given time-step (N=K), the user's response to the displayed images.

According to some embodiments, the device further comprising an apparatus selected from: a computer, a smart phone, a tablet and anu combination thereof.

According to some embodiments, the device further comprising at least one of: data storage for said user's inputs and provided analyses, an input device, a speaker device.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A, 1B, 1C and 1D schematically illustrate examples of visual acuity tests, using: letters, lines, or other objects with testing gaps;

FIGS. 2A and 2B schematically illustrate an original image (FIG. 2A), and a blurred image (FIG. 2B) comprising the original image (of FIG. 2A) applied with a blur level B, according to some embodiments of the invention;

FIGS. 3A, 3B and 3C schematically illustrate three blurred images comprising the same original image, yet applied with different levels of blur (B_{Fig.3B}>B_{Fig.3A}>B_{Fig.3C}), according to some embodiments of the invention;

FIGS. 4A-4B, 4C-4D, and 4E-4F schematically illustrate three different time steps (respectively N=1, 2, 3), with three different sets of images, of a displaying session, according to some embodiments of the invention;

FIG. 5 schematically illustrates devices and some of the method steps for displaying images, according to some embodiments of the invention;

FIG. 6 schematically illustrates devices and some of the method steps for displaying images, according to some embodiments of the invention; and

FIG. 7 demonstrates users' training test results, according to some embodiments of the invention.

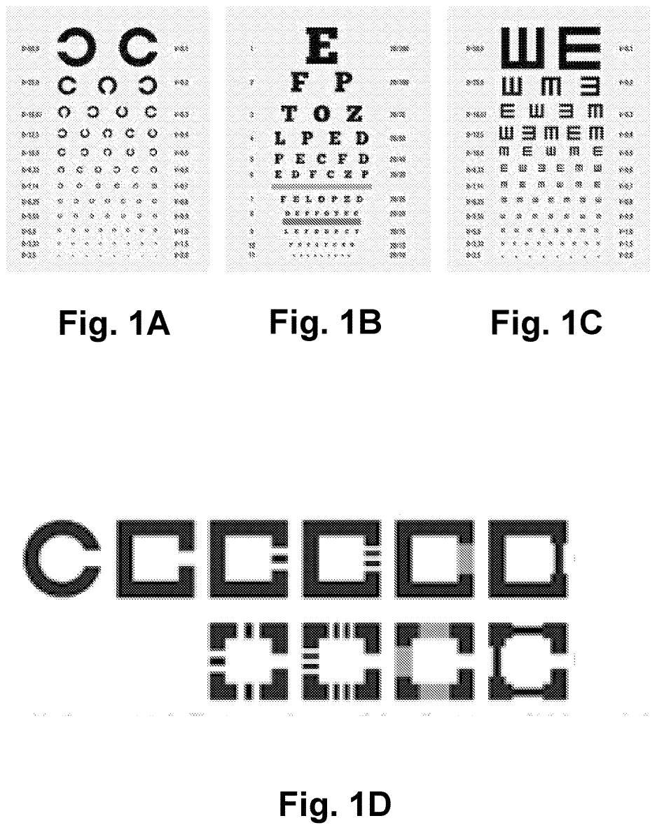

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

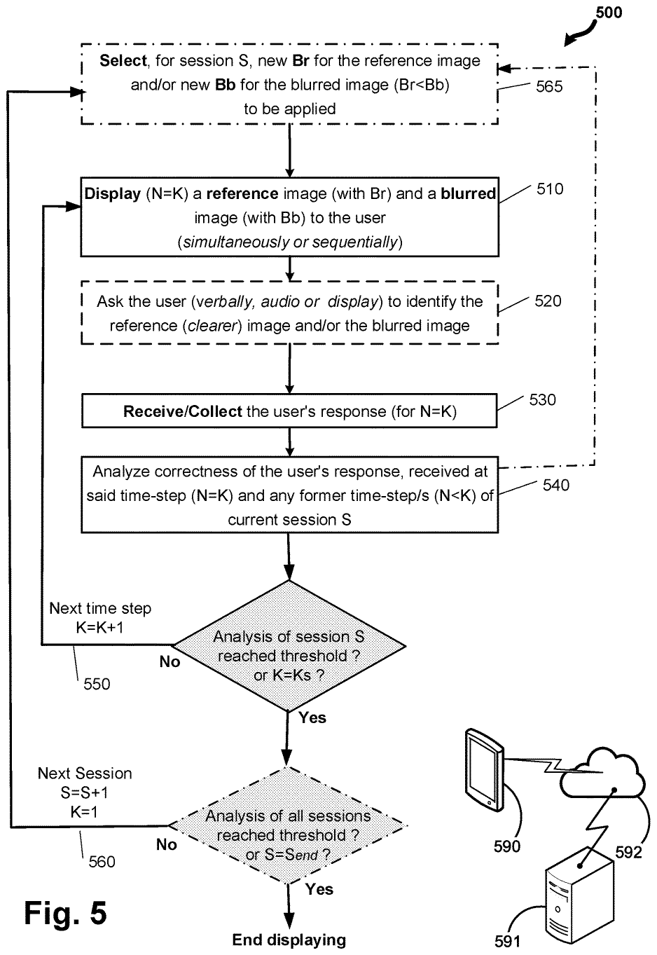

According to some embodiments of the invention, a new method is provided for presenting images to a user or a subject. In some embodiments, the terms “user” and “subject” may be used interchangeably having the same meanings. The method comprising at least one session S (S=1, 2, . . . . S_{end}) of providing sets of images (wherein each set comprises at least two images), at different time steps of said session, i.e., each set of images is provided at a different time step N (N=K, K=1, 2, . . . . Kₛ). Some of the method steps are demonstrated in FIG. 5.

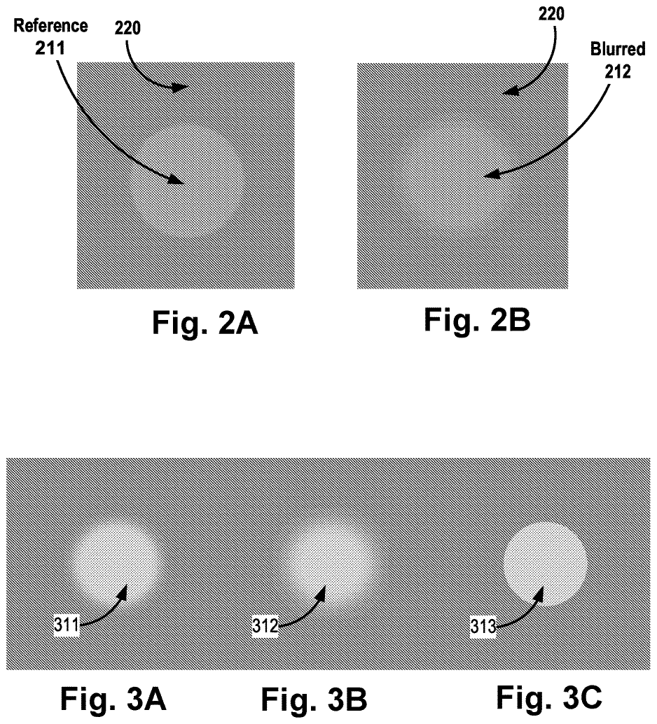

According to some embodiments, the method comprising processor implemented method steps of:
A. Displaying [510], at a given time-step N (N=K, 1≤K≤Kₛ) of a session S (S=1, 2, . . . . S_{end}) of time steps, a set of images to the user; the set comprising: at least one reference image and at least one blurred image; wherein both the reference- and the blurred-images comprise the same original image with different predetermined levels of blurriness (B) applied thereon (respectively Bᵣ and B_{b}), which are selected per said session S, and wherein Bᵣ<B_{b}. One example for a set of images, at a given time step, is provided in FIGS. 2A and 2B. FIG. 2A demonstrates a reference image [211], which is relatively clearer or less blurred than the blurred image as in FIG. 2A (B₂₁₁<B₂₁₂).
B. Receiving and/or Collecting [530], at said given time-step (N=K), the user's response to the currently displayed set of images, wherein the response comprises the user's indication of the reference (clearer) image and/or the blurred image. Clarification: according to some embodiments, the subject is requested to identify and/or indicate which of the presented images, at said time-step is clearer and/or which is more blurred.
C. Analyzing correctness of the user's response, received/collected at said given time-step (N=K) and any former time-step/s (N<K) of said session S. Reminder: all sets images of the time steps of a session are applied with same selected levels of blurriness Bᵣ and B_{b}).
D. Repeating method steps A to C with following time-steps (K=K+1) of said session S, a predetermined number of times (Kₛ) for said session(S) and/or until the correctness analysis of method steps C has reached a predetermined correctness threshold.

According to some embodiments, the method further comprising method step E of repeating method step D, with at least one following session (S=S+1) of time-steps (N=K, 1≤K≤Kₛ), while applying the reference image and/or the blurred image with a different level/s of blurriness (Bᵣ<B_{b}) for said following session [565]. According to some embodiments, the new level/s of the predetermined blurriness (Bᵣ and/or B_{b}) is/are selected respective to the analysis results of the former method step of analyzing C.

According to some embodiments, the method further comprising repeating method step E a predetermined number of times (i.e., predetermined number of sessions S_{end}) and/or until a comparison analysis of all former sessions has reached a predetermined analysis threshold.

According to some embodiments, method step E is provided with a predetermined time gap, i.e., a predetermined time break between sessions; e.g., a selected number of minutes, a selected number of hours, a selected number of days, a selected number of weeks, or any combination thereof.

According to some embodiments, at method step E [565], the newly selected level/s of predetermined blurriness (Bᵣ and/or B_{b}) for a next session is/are selected with a smaller gap between the values of Bᵣ and B_{b}; therefore, more difficult to identify the difference between the reference—and the blurred image. According to some embodiments the newly selected level/s of predetermined blurriness (Bᵣ and/or B_{b}) for a next sessions are selected with a smaller and smaller gaps between the values of Bᵣ and B_{b} (for example in staircase method) of until Bᵣ=B_{b}, then the expected answer is guessing (chance level).

According to some embodiments, method step E comprises selecting B_{b}, at one or more sessions, which is the user's current blurriness threshold (B_{b}=Bₜₕ); i.e., the user's tested limitation value that one can distinguish between clear vs. blurred images if (B≥Bₜₕ), and cannot distinguish therebetween if (B<Bₜₕ).

According to some embodiments, the method further comprising a method step or steps of testing the user, to determine one's current blurriness threshold Bₜₕ, at which the user's responses are correct a predetermined percentage of time steps. According to some embodiments, the testing threshold is selected as a predetermined percentage of correct responses; some non-limiting examples include a user being correct about 70%, 75%, 80%, 85% or any other selected percentage of the responses. According to some embodiment, the testing can be provided before and/or after every session, or number of sessions.

According to some embodiments, the user is provided with a real-time feedback of one's correctness, at said given time step (N=K).

According to some embodiments, the displaying of the set of reference-vs. blurred-images, at said given time step (N=K) is provided either simultaneously (e.g., side by side) or sequentially (e.g., one after the other; only one image at a time, yet same “time step”).

According to some embodiments, the original image comprise a round shape. According to some embodiments, the original image comprises no sharp edges.

According to some embodiments, and as demonstrated in FIGS. 3A-3C, the blurred level B is set and applied by a Gaussian variable Sigma.

According to some embodiments, mathematically, applying a Gaussian blur to an image is the same as convolving the image with a Gaussian function. This is also known as a two-dimensional Weierstrass transform. By contrast, convolving by a circle (i.e., a circular box blur) would more accurately reproduce the bokeh effect. Since the Fourier transform of a Gaussian is another Gaussian, applying a Gaussian blur has the effect of reducing the image's high-frequency components; a Gaussian blur is thus a low pass filter.

According to some embodiments, the Gaussian blur is a type of image-blurring filter that uses a Gaussian function (which also expresses the normal distribution in statistics) for calculating the transformation to apply to each pixel in the image. The formula of a Gaussian function in one dimension is:

$$G ( x ) = \frac{1}{\sqrt{2 {πσ}^{2}}} {e}^{- \frac{{x}^{2}}{2 {σ}^{2}}}$$

In two dimensions, it is the product of two such Gaussian functions, one in each dimension:

$$G ( x , y ) = \frac{1}{\sqrt{2 {πσ}^{2}}} {e}^{- \frac{{x}^{2} + {y}^{2}}{2 {σ}^{2}}}$$

where x is the distance from the origin in the horizontal axis, y is the distance from the origin in the vertical axis, and σ is the standard deviation of the Gaussian distribution. When applied in two dimensions, this formula produces a surface whose contours are concentric circles with a Gaussian distribution from the center point. According to some embodiments, values from this distribution are used to build a convolution matrix which is applied to the original image.

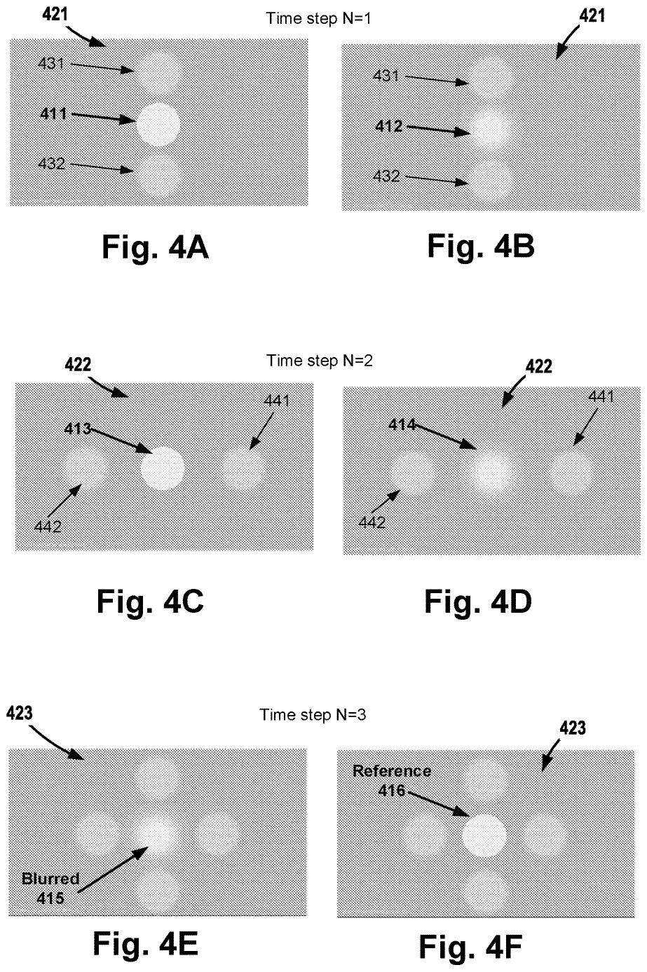

According to some embodiments, and as demonstrated in FIGS. 2A-2B, 4A-4B, 4C-4D and 4E-4F, both the reference- and the blurred-images, at each time step (N=K), are displayed with same predetermined background features (220, FIGS. 2A-2B), such that the only difference is the reference-[211] vs. blurred-image [212]. Background features are selected from: contrast, colors, brightness, texture and other background images (not to be tested by) such as but not limited to sea view or forest and such.

An example for three different time steps, with three different sets of displayed images (reference vs. blurred), of a session (S=1), is provided in FIGS. 4A-4B (for N=1), FIGS. 4C-4D (for N=2) and FIGS. 4E-4F (for N=3), where blurriness level Br<Bb is fixed for all the three time steps. According to some embodiments, and as demonstrated in FIGS. 4A-4F, the original image is same for all time steps (in this example a simple filled circle). According to such an embodiment, the original image per each session (S=1, 2 . . . . S_{end}) can be different or kept same. According to some embodiments, the original image per each time step of a session can be different (not shown), while Br<Bb are fixed for said session. FIGS. 4A-4B (for N=1), FIGS. 4C-4D (for N=2) and FIGS. 4E-4F (for N=3) further demonstrate that the background is identical per a set of images of a given time step, i.e., FIGS. 4A-4B with background [421], FIGS. 4C-4D with background [422], and FIGS. 4E-4F with background [423]. It is further demonstrated that the background can comprise background-image/s which may be clear or blurred, e.g., [431,432] as in FIGS. 4A-4B and [441,442] as in FIGS. 4C-4D, these background images are not to be identified or indicated.

According to some embodiments the method further comprises instructing the user to identify and/or indicate, which of the images is clearer, or which of the images is more blurred, or both. According to some embodiments, the instruction is general and same for all sessions and all time-steps. According to some embodiments, the instruction can be changed per each session. According to some embodiments, the instruction can be changed per each time step. According to some embodiments the instruction may be provided to the user verbally, by a monitoring personal. According to some embodiments the instruction may be provided to the user via a processor and at least one device selected from: a display device, a speaker.

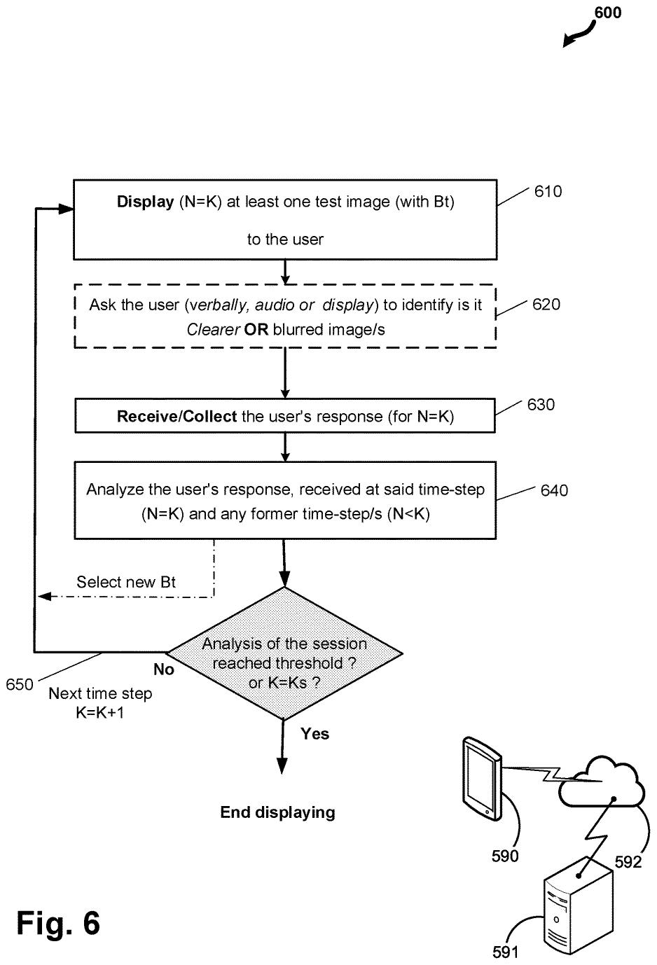

According to some embodiments of the invention, a new method is provided for presenting images to a user or a subject. In some embodiments, the terms “user” and “subject” may be used interchangeably having the same meanings. The method comprising at least one session of providing at least one test image, at different time steps of said session, i.e., test image/s is/are provided at a different time step N (N=K, K=1, 2, . . . . Kₛ). Some of the method steps are demonstrated in FIG. 6.

A method for presenting images to a user, the method comprising processor implemented method steps of:
a. displaying [610], at a given time-step N (N=K, 1<K<Kₛ) of a session of time steps, at least one test image; wherein each of the test images comprises its own original image with a predetermined level of blurriness applied thereon Bₜ, selected per said time step (N=K); clarification: Bₜ is same for all images at said time step (N=K).
b. receiving and/or collecting [630], at said given time-step (N=K), the user's response to the currently displayed images, wherein the response comprises the user's indication whether the at least one test image is clear or blurred; clarification: a single (one) answer for all test images presented at said time-step (N=K).
c. analyzing the user's response, received at said given time-step (N=K) and any former time-step/s (N<K) of said session;
d. repeating method steps a to c with following time-steps (K=K+1) of said session, a predetermined number of times Kₛ for said session and/or until the analysis of method step c has reached a predetermined threshold; wherein the selected Bₜ for the following time-step is based on the analysis of step c.

According to some embodiments, method step d is provided with a predetermined time gap, i.e., a predetermined time break between sessions; e.g., a selected number of minutes, a selected number of hours, a selected number of days, a selected number of weeks, or any combination thereof.

According to some embodiments, method step d comprises selecting Bₜ which is the user's current blurriness threshold (Bₜ=Bₜₕ).

According to some embodiments, the method further comprising testing the user to determine one's current blurriness threshold Bₜₕ, at which the user's responses are correct a predetermined percentage of time steps. According to some embodiments, the testing threshold is selected as a predetermined percentage of correct responses; some non-limiting examples include a user being correct about 70%, 75%, 80%, 85% or any other selected percentage of the responses. According to some embodiment, the testing can be provided before and/or after every time-step, or before and/or after a session.

According to some embodiments, the user is provided with a real-time feedback of one's correctness, at said given time step (N=K).

According to some embodiments, the original/s image comprise a round shape.

According to some embodiments, the original/s image comprise no sharp edges.

According to some embodiments, the blurred level B is set by a Gaussian variable Sigma.

According to some embodiments, in the case of more than one test image, all test images at said time step (N=K), are displayed with same predetermined features selected from: background, contrast, colors, brightness, texture, other images.

According to some embodiments, the original image changes between the different time steps and/or wherein the predetermined selected features change between the different time steps.

According to some embodiments the method further comprises instructing the user to indicate if the test image/s is/are clear, or if the test image/s are blurred. According to some embodiments, the instruction is general and same for all sessions and all time-steps. According to some embodiments, the instruction can be changed per each session. According to some embodiments, the instruction can be changed per each time step. According to some embodiments the instruction may be provided to the user verbally, by a monitoring personal. According to some embodiments the instruction may be provided to the user via a processor and at least one device selected from: a display device, a speaker.

According to some embodiments a method is provided for treating a subject afflicted with Myopia and/or Age-related Macular Degeneration (AMD), the method comprising presenting images to the subject according to any one of the above-mentioned method steps.

According to some embodiments, a new device is provided, configured to provide images to a user, the device comprising:
at least one processor [590,591,592] configured to implement the method steps according to any one of the above-mentioned method steps;
at least one display-device [590], configured to display the images to the user; and
at least one input-device [590], configured to receive and/or collect and interpret, at the given time-step (N=K), the user's response to the displayed images.

According to some embodiments, the device comprising an apparatus selected from: a computer [591], a smart phone, a tablet [590], and any other device or system comprising a display, an input collector and a processor.

According to some embodiments, the new device and methods according to any one of the above embodiments are configured for training a user for improving ones blurred vision and/or visual acuity. According to some of such embodiments, the method is configured for training—and/or help vision correction of—patients suffering from: Myopia and Age-related Macular Degeneration (AMD).

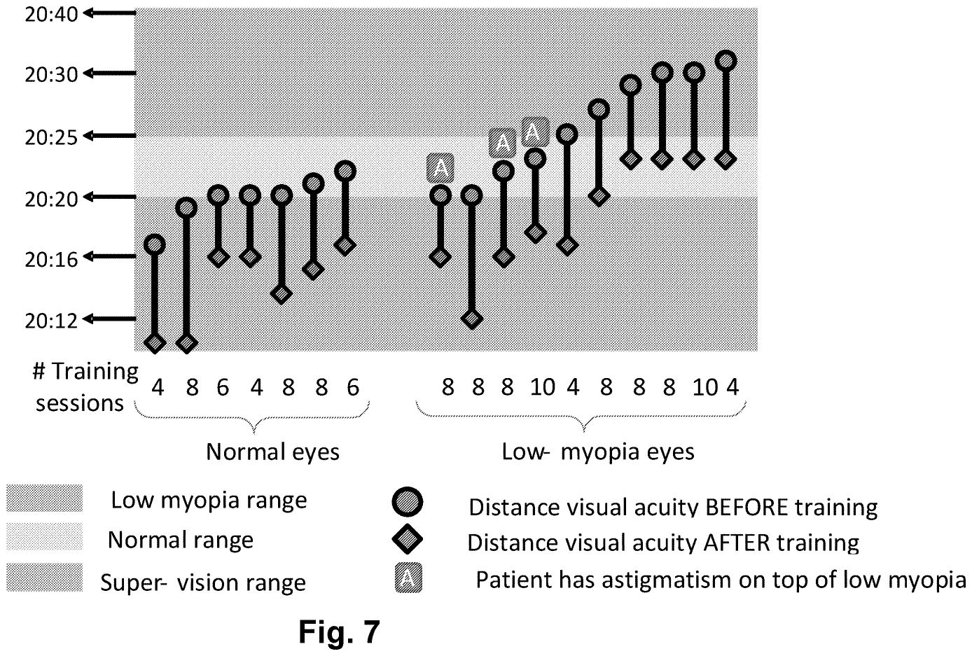

Reference is made to FIG. 7, which demonstrates training test results. As shown, each vertical line indicates a different trained eye. Seventeen different eyes were trained and tested; some may be of same person. The circle of each vertical line demonstrates the eye status before training and the diamond the eye's condition after several training sessions, (indicated below at the X-axis), where the Y-axis demonstrates the eye's vision acuity. As shown, visual acuity (USCS) improvement was achieved after 4-8 training sessions for the seven normal eyes and improvement was also achieved after 4-10 training session of ten low-myopia eyes.

TABLE 1
Near visual acuity improvement following
treatment, dry-stage AMD patients

| | Male/ | | # | Post treatment |
|---|---|---|---|---|
| Age | Female | Clinical baseline | Sessions | improvement |
| 86 | Male | Left: Mild AMD | 12 | Left: 45% |
| | | Right: Moderate AMD | | Right: 74% |
| 76 | Female | Left: Mild AMD | 30 | Left: 82% |
| | | Right: Mild AMD | | Right: 58% |
| 71 | Male | Left: Mild AMD | 12 | Left: Excluded* |
| | | Right: Mild AMD | | Right: 58% |

*Left eye excluded as patient's cataract in the left eye worsened during the training period and vision in that eye deteriorated significantly

Reference is made to Table 1, which demonstrates training test results of near visual acuity, following treatment, for dry-stage AMD patients. As shown, six different eyes of three subjects were trained and tested. The “Clinical baseline” column demonstrates the eye status before training and the “Post treatment improvement” demonstrates the improvement after 12-30 training sessions. As shown, visual acuity (USCS) improvement was achieved after 12-30 training sessions for dry-stage AMD eyes.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for presenting images to a user, the method comprising processor implemented method steps of:
A. displaying, at a given time-step N (N=K, 1≤K≤Kₛ) of a session S of time steps, at least one reference image and at least one blurred image to the user; wherein both the reference and the blurred image comprise the same original image with different predetermined levels of blurriness applied thereon Bᵣ, B_{b}, selected per said session S; and wherein Bᵣ<B_{b};
B. receiving and/collecting, at said given time-step (N=K), the user's response to the currently displayed images, wherein the response comprises the user's indication of the reference image and/or the blurred image;
C. analyzing correctness of the user's response, received at said given time-step (N=K) and any former time-step/s (N<K) of said session S;
D. repeating method steps A to C with following time-steps (K=K+1) of said session S, a predetermined number of times Kₛ for said session S and/or until the correctness analysis of method step C has reached a predetermined correctness threshold.

2. The method of claim 1, further comprising method step E of repeating method step D, with at least one following session (S=S+1) of time-steps (N=K, 1≤K≤K_{S}), while applying the reference image and/or the blurred image with a different level/s of blurriness (Bᵣ<B_{b}) for said following session.

3. The method of claim 2, wherein the new level/s of predetermined blurriness (Bᵣ and/or B_{b}) is/are selected respective to the analysis results of the former method step/s C.

4. The method of claim 2, the method further comprises repeating method step E a predetermined number of times and/or until a comparison analysis of all former sessions has reached a predetermined analysis threshold.

5. The method of claim 4, wherein method step E is provided with a predetermined time gap.

6. The method of claim 2, wherein the new level/s of predetermined blurriness (Bᵣ and/or B_{b}) is/are selected with a smaller gap between Bᵣ and B_{b}.

7. The method of claim 2, wherein method step E comprises selecting B_{b} which is the user's current blurriness threshold (B_{b}=Bₜₕ).

8. The method of claim 7, further comprising testing the user to determine one's current blurriness threshold Bₜₕ, at which the user's responses are correct a predetermined percentage of time steps.

9. The method of claim 1, wherein the user is provided with a real-time feedback of one's correctness, at said given time step (N=K).

10. The method of claim 1, wherein the displaying of the reference vs. blurred images is provided either simultaneously or sequentially.

11. The method of claim 1, wherein the original image comprises a round shape.

12. The method of claim 11, wherein the original image comprises no sharp edges.

13. The method of claim 1, wherein the blurred level B is set by a Gaussian variable Sigma.

14. The method of claim 1, both the reference and blurred images, at each time step (N=K), are displayed with same predetermined features selected from: background, contrast, colors, brightness, other images.

15. The method of claim 14, wherein the original image changes between the different time steps and/or wherein the predetermined selected features change between the different time steps.

16-25. (canceled)

26. The method of claim 1, used for improving visual acuity of a subject afflicted with Myopia and/or Age-related Macular Degeneration (AMD.

27. A device configured to present images to a user, comprising:
at least one processor configured to implement the method steps according to claim 1;
at least one display-device, configured to display the images to the user; and
at least one input-device, configured to collect and interpret, at the given time-step (N=K), the user's response to the displayed images.

28. The device of claim 27, further comprising an apparatus selected from: a computer, a smart phone, a tablet and anu combination thereof.

29. The device of claim 27, further comprising at least one of: data storage for said user's inputs and provided analyses, an input device, a speaker device.
